(19) United States
(12) Patent Application Publication
(10) Pub. No.: US 2025/0380853 A1
(43) Pub. Date: Dec. 18, 2025

(54) SEAL FOR AN ENDOSCOPE

(71) Applicant: Boston Scientific Scimed, Inc., Maple Grove, MN (US)

(72) Inventors: Nathan Thomas Cummings, Worcester, MA (US); Tess Davis, Boston, MA (US)

(73) Assignee: Boston Scientific Scimed, Inc., Maple Grove, MN (US)

(21) Appl. No.: 19/240,324

(22) Filed: Jun. 17, 2025

Related U.S. Application Data
(60) Provisional application No. 63/661,217, filed on Jun. 18, 2024.

(51) Int. Cl. A61B 1/00

(52) U.S. Cl. CPC A61B 1/00068; A61B 1/00137

(57) ABSTRACT

Devices, systems, and methods for a valve assembly for a medical device. The valve may include a valve stem and a valve body. The valve stem may include an elongate body, a first opening in the elongate body, a second opening in the elongate body, a lumen extending between the first opening and the second opening, and a seal extending circumferentially around the elongate body. The seal may include a body portion, a flange extending circumferentially around the elongate body and radially outward from the elongate body, and one or more ribs extending between the body portion and the flange.

### CROSS-REFERENCE TO RELATED APPLICATIONS

This application claims the benefit of U.S. Provisional Patent Application Ser. No. 63/661,217 filed on Jun. 18, 2024, the disclosure of which is incorporated herein by reference.

### TECHNICAL FIELD

This disclosure relates generally to valve assemblies and methods, and particularly to seals for valves of endoscopes and methods for an endoscope

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a valve stem for a medical device may include an elongate body, a first opening in the elongate body, a second opening in the elongate body, a lumen extending between the first opening and the second opening, and a seal extending circumferentially around the elongate body, the seal having a body portion, a flange extending circumferentially around the elongate body and radially outward from the elongate body, and one or more ribs extending between the body portion and the flange.

Alternatively or additionally to any of the examples above, the seal may include a plurality of ribs equally spaced circumferentially around the body portion of the seal.

Alternatively or additionally to any of the examples above, the flange may have a first side and a second side opposing the first side and the one or more ribs may extend between the body portion and the first side of the flange, and the one or more ribs may be configured to be in tension and maintain the flange in a taut configuration when a pressure at the first side of the flange is greater than a pressure at the second side of the flange.

Alternatively or additionally to any of the examples above, the one or more ribs may be configured to flex and allow the flange to deform when the pressure at the second side of the flange is greater than the pressure at the first side of the flange.

Alternatively or additionally to any of the examples above, the one or more ribs may comprise a first set of one or more ribs each having a first rigidity and a second set of one or more ribs each having a second rigidity that is more rigid than the first rigidity.

Alternatively or additionally to any of the examples above, the first set of one or more ribs may have more ribs than the second set of one or more ribs.

Alternatively or additionally to any of the examples above, the second set of one or more ribs may have a plurality of ribs equally spaced circumferentially around the body portion of the seal and the first set of one or more ribs may have a plurality of ribs equally divided between circumferentially sequential ribs of the second set of one or more ribs.

Alternatively or additionally to any of the examples above, the flange may have a first side and a second side opposing the first side and the first set of one or more ribs and the second set of one or more ribs may extend between the body portion and the first side of the flange, and the ribs of the first set of one or more ribs may be configured to flex and allow the flange to deform between the sequential ribs of the second set of one or more ribs when a pressure at the second side of the flange is greater than a pressure at the first side of the flange.

In another example, a seal for a valve stem of a medical device may include a body portion defining a lumen configured to receive the valve stem, a flange extending circumferentially around the body portion, and a plurality of ribs extending between the body portion and the flange.

Alternatively or additionally to any of the examples above, the plurality of ribs may be equally spaced circumferentially around the body portion.

Alternatively or additionally to any of the examples above, each rib of the plurality of ribs may have a rigidity configured to maintain the flange in a taut configuration when a pressure at a first side of the flange is greater than a pressure at a second side of the flange and allow the flange to deform when the pressure at the second side of the flange is greater than the pressure at the first side of the flange.

Alternatively or additionally to any of the examples above, the plurality of ribs may comprise a first set of one or more ribs each having a first rigidity and a second set of one or more ribs each having a second rigidity that is more rigid than the first rigidity.

Alternatively or additionally to any of the examples above, the first set of one or more ribs may have more ribs than the second set of one or more ribs.

Alternatively or additionally to any of the examples above, the second set of one or more ribs may have a plurality of ribs equally spaced circumferentially around the body portion of the seal and the first set of one or more ribs may have a plurality of ribs equally divided between circumferentially sequential ribs of the second set of one or more ribs.

Alternatively or additionally to any of the examples above, the flange may have a first side and a second side opposing the first side and the first set of one or more ribs and the second set of one or more ribs may extend between the body portion and the first side of the flange, and the ribs of the first set of one or more ribs may be configured to flex and allow the flange to deform between the ribs of the second set of one or more ribs when a pressure at the second side of the flange is greater than a pressure at the first side of the flange.

Alternatively or additionally to any of the examples above, the first set of one or more ribs may have nine ribs, the second set of one or more ribs may have three ribs equally spaced circumferentially about the body portion, and three ribs of the first set of one or more ribs may be positioned between each two circumferentially sequential ribs of the second set of one or more ribs.

In another example, a valve assembly for a medical device may include a valve body having a gas inlet passage and a gas outlet passage, a valve cap proximate a proximal end of the valve body, a valve stem connected to the valve cap and configured to translate within the valve body between an upper position and a lower position, the valve stem comprising an elongate body and a central lumen extending through the elongate body between a first opening in the elongate body and a second opening in the elongate body, and a seal extending around the valve stem, the seal having a flange and one or more ribs supporting the flange in a flow blocking configuration between the gas inlet passage and the gas outlet passage.

Alternatively or additionally to any of the examples above, the flange may have a first side and a second side, the one or more ribs may extend between a body portion of the flange and the first side, the one or more ribs may be configured to support the flange in the flow blocking configuration when a pressure on the first side of the flange is greater than a pressure on the second side of the flange, and the one or more ribs may be configured to flex to allow the flange to adjust to a flow passing configuration when a pressure on the second side of the flange is greater than a pressure on the first side of the flange.

Alternatively or additionally to any of the examples above, the one or more ribs may comprise at least one rib with a first rigidity and at least one rib with a second rigidity that is more rigid than the first rigidity.

Alternatively or additionally to any of the examples above, the seal may be located at a position along the valve stem that is configured to be maintained between the air inlet passage and the air outlet passage of the valve body during operation of the medical device.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a schematic perspective view of an illustrative valve;

FIG. 4A depicts a schematic cross-section view of an illustrative valve, with the valve in a first configuration;

FIG. 4B depicts a schematic cross-section view of the illustrative valve of FIG. 4A, with the valve in a second configuration;

FIG. 4C depicts a schematic cross-section view of the illustrative valve of FIG. 4A, with the valve in a third configuration;

FIG. 5A depicts a schematic cross-section view of an illustrative valve, with the valve in the first configuration;

FIG. 5B depicts a schematic cross-section view of the illustrative valve of FIG. 5A, with the valve in the second configuration;

FIG. 6A depicts a schematic top perspective view of an illustrative seal;

FIG. 6B depicts a schematic bottom perspective view of the illustrative seal of FIG. 6A;

FIGS. 7A and 7B depict schematic cross-section views of an illustrative valve in the first configuration and the second configuration of the valve, respectively; and

FIGS. 8A and 8B depict schematic cross-section views of an illustrative valve in the first configuration and the second configuration of the valve, respectively.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it would be within the knowledge of one skilled in the art to effect the particular feature, structure, or characteristic in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. The numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element.

The detailed description is intended to illustrate but not limit the disclosure. The various elements described may be arranged in various combinations and configurations without departing from the scope of the disclosure. The detailed description illustrates example configurations of the disclosure.

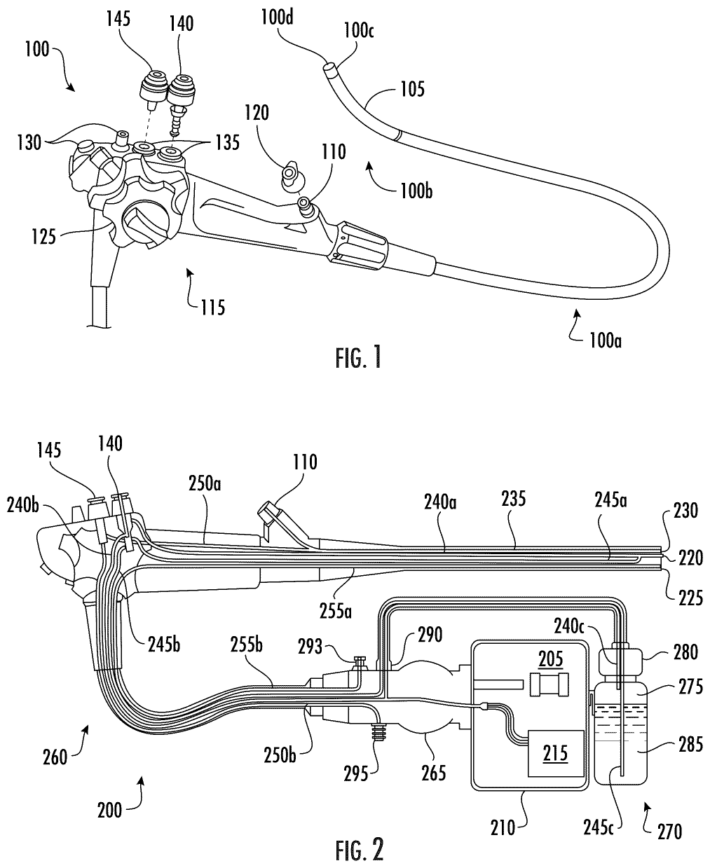

With reference to FIG. 1, an illustrative endoscope 100 is schematically depicted and FIG. 2 schematically depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump (e.g., an air pump 215), such as an air feed pump, in the unit 210.

The endoscope shaft 100a may include a distal tip 100c (e.g., a distal tip unit adapted to be inserted into a body cavity of a patient) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion 105 (e.g., one knob controls up-down steering and another knob controls left-right steering). One or more (e.g., one, two, a plurality, etc.) of video switches 130 for remotely operating the video processing unit 210 may be arranged on a proximal end side of the handle 115.

The handle 115 may be provided with dual valve locations 135. One of the valve locations 135 may have or receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220, as depicted in FIG. 2.

The other valve location 135 may have or receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water container or reservoir 270 (e.g., a water bottle and/or other suitable reservoir or container) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

Referring to FIG. 2, an illustrative operation of an endoscope system 200, including an endoscope such as the endoscope 100 depicted in FIG. 1, is explained. Air from the air pump 215 in the video processing unit 210 may flow through the connector portion 265 and branch to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve 140 to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve 140, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some configurations, outflow check valves or other one-way valve configurations (not shown) may be placed in the path of the lens wash supply tubing to help prevent water from back-flowing into the reservoir 270 after the water has passed the valve.

A relatively higher flow rate compared to lens wash is typically required for irrigation water, since a primary use is to clear the treatment area in the patient of debris that obstructs the user's field of view. Irrigation is typically achieved with the use of a pump (e.g., peristaltic pump), as described. In configurations with an independent water source for irrigation, tubing placed in the bottom of a water source may be passed through the top of the water source and threaded through the head on the upstream side of the pump. Tubing on the downstream side of the pump 255c is connected to the irrigation feed line 255b in the umbilical 260 and the irrigation supply line 255a of endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical 260, and down the irrigation supply line in the shaft 100a of the endoscope 100 to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some configurations, outflow check valves or other one-way valve configurations (not shown), similar to the lens wash tubing 245c, may be placed in the path of the irrigation supply tubing to help prevent back-flow into the reservoir after water has passed the valve.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve 145 may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct a suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilical 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position)

Gas/water valves 140 and/or other valves (e.g., the suction valve 145, etc.) may include one or more one-way seals extending around a valve stem of the valve. The one or more one-way seals may be configured to prevent backflow through the valves. In one example in which the gas/water valve 140 includes a one-way seal, the one-way seal may be utilized to maintain insufflation pressure in an active lumen of the endoscope 100.

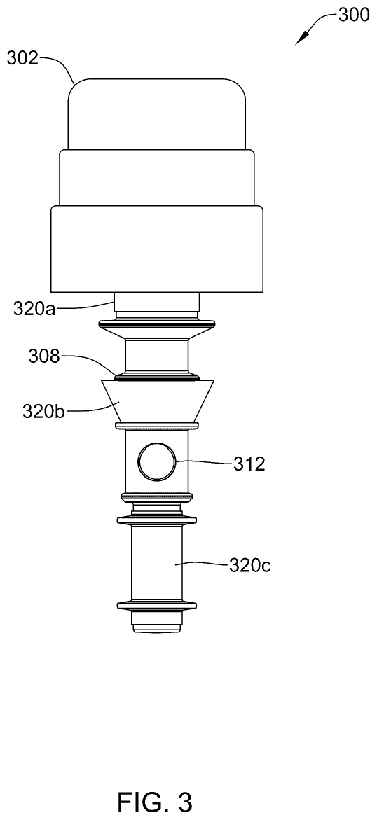

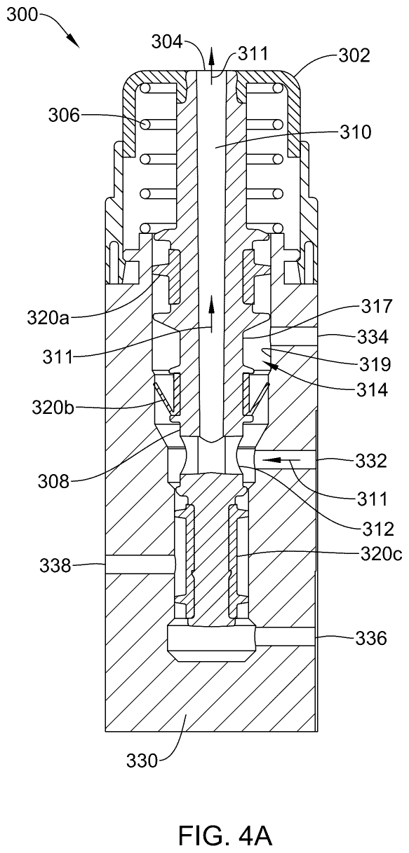

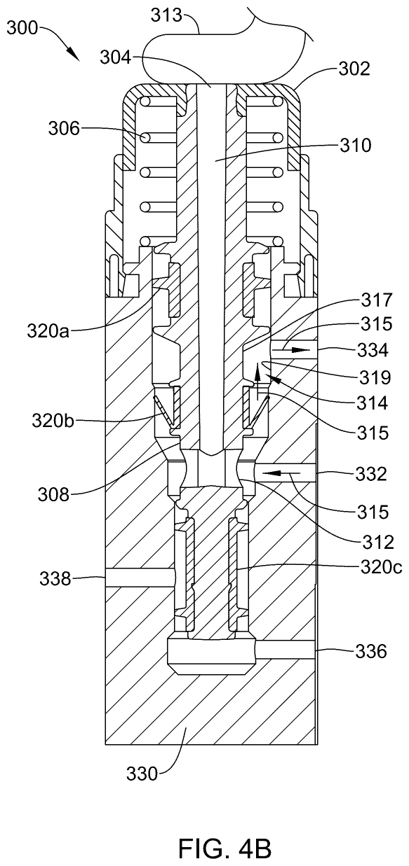

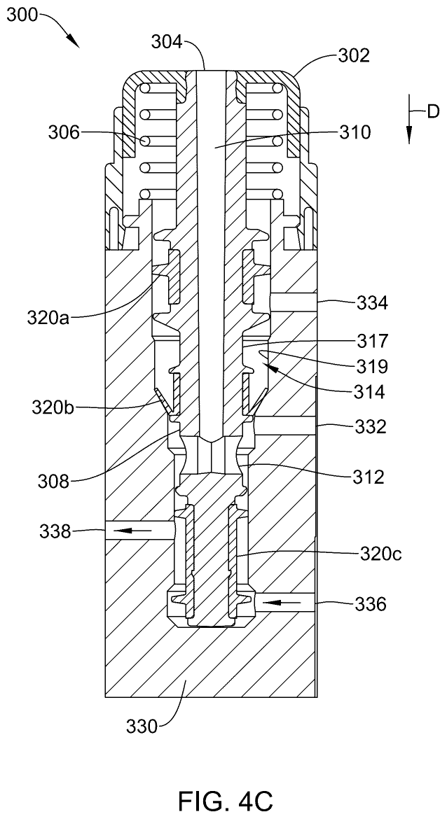

An example configuration of the gas/water valve (e.g., the gas/water valve 140 or other suitable gas/water valve) including a one-way seal is depicted in FIGS. 3 and 4A-4C as a gas/water valve 300. Among other suitable components and/or features, the gas/water valve 300 may include a valve cap 302, a spring member 306, and a valve stem 308. The valve cap 302 may include one or more openings (e.g., a gas escape hole and/or other suitable opening) and the valve stem 308 may include a central lumen 310 extending between one or more first openings 304 (e.g., one or more first openings in communication with at least one of the one or more openings of the valve cap 302) and one or more second openings 312 (e.g., a gas inlet passage).

The valve stem 308 may couple to the cap 302 in any suitable manner. In some cases, a portion (e.g., a proximal portion) of the valve stem 308 extending proximally of the opening 304 may be coupled to the cap 302 via one or more suitable coupling mechanisms. Example suitable coupling mechanisms include, but are not limited to, adhesives, a threaded connection, a luer lock connection, a snap connection, a ball-detent connector, a friction fit, and/or additional or alternative coupling mechanisms.

The valve stem 308 may have any suitable configuration. In some examples, the valve stem 308 may be configured to adjust positions within the valve well, adjust flow paths to the gas and liquid supplies and feeds, and couple to the cap 302.

The valve stem 308 may be formed in any suitable manner. In some examples, though not required, the valve stem 308 may be formed using a molding process, an injection molding process, a casting process, a finishing process, sanding, and/or by or with one or more additional or alternative manufacturing techniques. In one example, the valve stem 308 may be formed using an injection molding process.

The valve 300 may include any suitable number of seals along the valve stem 308. In some examples, three seals 320a, 320b, 320c may extend circumferentially around (e.g., extend partially or entirely circumferentially around) the valve stem 308 (e.g., around the elongate body of the valve stem 308) at different axial locations. In one example, a first seal 320a may be located between the first opening 304 and the second opening 312 at a location configured to be proximal of the gas outlet passage 334 to obstruct flow in the valve 300 proximal the location of the gas outlet passage 334, a second seal 320b may be located between the first seal 320a and the second opening 312 and may be configured to not impede gas flow when the valve stem 308 is in the configuration (e.g., as depicted in FIG. 4B), and a third seal 320c may be located between a distal end of the valve stem 308 and the gas inlet 312, but other suitable configurations are contemplated. Alternatively or additionally, the seals of the valve 300 may be located along the valve body 330 such that the valve stem 308 may move relative to the seals.

The seals 320a, 320b, 320c may have any suitable configuration. In some examples, each of the seals 320a, 320b, 320c may include on one or more wipers or flanges configured to engage the inner surface 319 of the valve body 330. In one example, the first seal 320a may have a single wiper or flange, the second seal 320b may have a single wiper or flange, and the third seal 320c may have two wipers or flanges, but other suitable configurations are contemplated.

The components of the valve 300 may be formed from any suitable materials. In some examples, the valve stem 308 may be formed from a first material and seals 320a-320c of or coupled with the valve stem 308 may be formed from a second material, where the second material may be the same as or different than the first material. The valve stem 308 may be formed from a hard or rigid polymer and all of, part of one or more of, or one or more of the seals 320a-320c may be formed from a flexible polymer, but other suitable configurations are contemplated. In some examples, the valve stem 308 may be formed from polymer, acrylonitrile butadiene styrene (ABS), polycarbonate, and/or other suitable material. Alternatively, the valve stem 308 may be formed of steel or aluminum. The seals 320a-320c may be formed from one or more of a polymer, thermoplastic elastomers (TPE), thermoplastic polyurethane (TPU), liquid silicone rubber (LSR), and/or other suitable materials.

The material of the seals 320a, 320b, 320c may have any suitable durometer. In one example, the material of the seals 320a, 320b, 320c when positioned at the valve stem 308 may have a durometer in a range of about 20-80 shore A, about 30-60 shore A, and/or other suitable values within one or more other suitable ranges of durometer, but could be softer or firmer depending on the geometry used for the seals and the amount of interference desired with an inner surface 319 of a valve body 330 (e.g., depicted in FIGS. 4A-4C). In one example, the seals 320a, 320b, 320c may be formed from silicone with a durometer in a range of 40-50 shore A, but this is not required.

The valve 300 may be inserted into a valve body 330 (e.g., one of the valve locations 135 described with respect to FIGS. 1 and 2). The valve body 330 may be sized and shaped to receive the valve stem 308, as well as alternative valve designs (including, but not limited to, each of those illustrated and described herein). The valve body 330 may include a gas inlet passage 332 (e.g., an air inlet passage) communicating with a source of gas (e.g., air, etc. as described above with respect to the gas supply line 240a). A gas outlet passage 334 (e.g., an air outlet passage) in the valve body 330 may communicates with a gas feed line (e.g., the gas feed line 240b and/or other suitable gas line). The valve body 330 may include a liquid inlet passage 336 (e.g., a water inlet, etc.) connected to or otherwise in communication with a liquid supply (e.g., a water supply) and a liquid outlet passage 338 (e.g., a water outlet, etc.) connected to or otherwise in communication with a liquid feed line.

Gas flowing into the valve body 330 via the gas inlet passage 332, may travel along the path of least resistance and as such, the gas may flow along a first flow path when the opening 304 is unblocked (e.g., the valve 300 is in a first configuration) and along a second flow path (e.g., the valve 300 is in a second configuration) (including across the second seal 320b as discussed below) when the opening 304 is blocked. FIG. 4A schematically depicts the valve 300 in the first configuration with the opening 304 unblocked, which may allow gas to travel along the first flow path (e.g., represented by arrows 311) passing from and through the gas inlet passage 332, the second opening 312 of the valve stem 308, along the central lumen 310, and into the ambient through the first opening 304 of the valve stem 308. FIG. 4B schematically depicts the valve 300 in the second configuration with the first opening 304 obstructed. In some examples, a user may place a finger 313 (e.g., thumb or other suitable finger) over the opening 304 to block the first opening. A flap or other device or component may also be included for placement over the first opening 304 in additional and/or alternative configurations. When the opening 304 is obstructed or blocked, gas may travel along the second flow path (e.g., represented by arrows 315) from and through the gas inlet passage 332, a space 314 between an outer surface 317 of the valve stem 308 and the inner surface 319 of a side wall of the valve body 330, and through the gas outlet passage 334, and into a lumen of the endoscope 100 for use in insufflation.

FIG. 4C schematically depicts a third configuration of the valve 300 in which the valve stem 308 has been advanced distally relative to the valve body 330. In some examples, a force in a distal direction D on the valve cap 302 (e.g., via a finger of a user and/or other suitable actuator) may advance the valve stem 308 in the distal direction and maintain the valve stem 308 in the third configuration. When the force in the distal direction D is removed from the valve cap 302, a spring member 306 (e.g., a coil spring and/or other suitable mechanism configured to exert a spring or bias force) may return the valve cap 302 and the valve stem 308 to the first or second configuration. In the third configuration, as depicted for example in FIG. 4C, the space 314 is no longer in fluid communication with the gas inlet passage 332 in the valve body 330. Rather, the second seal 320b (e.g., a middle seal or other suitably positioned seal) may be seated along the inner wall of the body 330 to obstruct or prevent gas flow proximal of the gas inlet passage 332.

When the valve stem 308 is in the third configuration, the two wipers or flanges of the third seal 320c may be positioned such that a distal wiper or flange of the third seal 320c is distal of the liquid inlet passage 336 and a proximal wiper or flange of the third seal 320c is proximal of the liquid outlet passage 338, which may create an annular passage between the valve stem 308 and valve body 330 in which liquid may flow from the liquid inlet passage 336 to the liquid outlet passage 338. Upon release of the force in the distal direction D and return of the valve cap 302 and valve stem 308 to respective positions associated with the first or second configurations, the placement of the third seal 320c may block or prevent additional liquid from entering the space between the axially spaced wipers or flanges of the third seal 320c. In the first, second, or third configurations of the valve 300, the placement of the third seal 320c on the valve stem 308 may prevent liquid from entering gas flow paths.

The second seal 320b depicted in FIGS. 3-4C may have a cone-shape configuration. The cone-shape configuration may be capable of partially or fully inverting unintentionally during use as the valve stem 308 adjusts axially and/or as pressures between the valve stem 308 and the valve body 330 change during use of the valve 300. When the second seal 320b is inverted or inverts (e.g., partially or entirely inverts), the inversion may diminish an effectiveness of the seal to seal between the gas inlet passage 332 and the gas outlet passage 334 and may cause a force required to adjust a position of the valve stem 308 to increase relative to when the second seal 320b is not inverted.

To mitigate unintended inversion of the second seal 320b, a one-way valve with a flange portion supported by one or more ribs may be utilized. In some examples, the one or more ribs may have a desired strength, flexibility, and/or rigidity such that when tensioned (e.g., when in tension) the ribs cause the flange to prevent backflow or insufflation leakage across the flange and when compressed (e.g., when in compression) relative to when the ribs are tensioned allow fluid to travel from the gas inlet passage 332 to the gas outlet passage 334 (e.g., to allow insufflation).

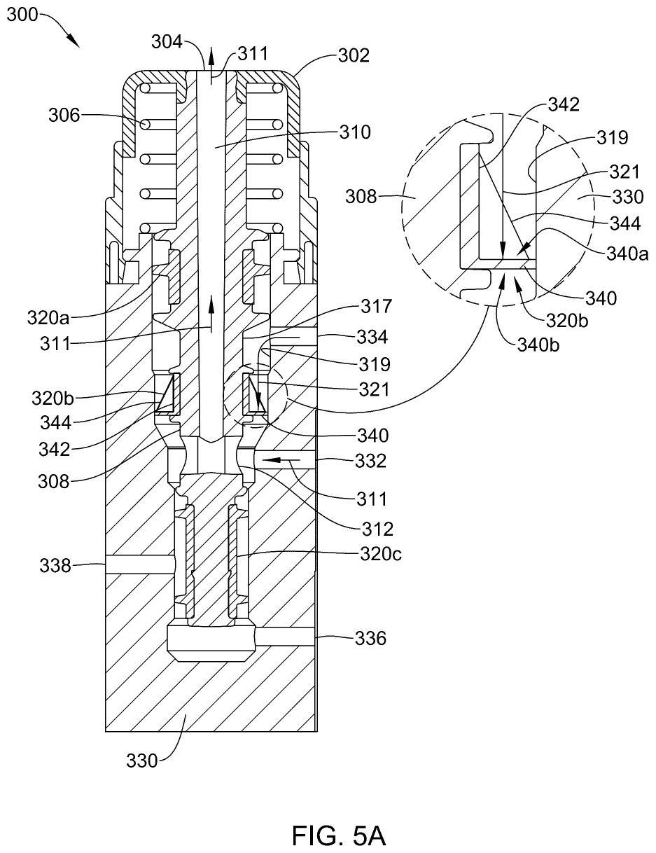

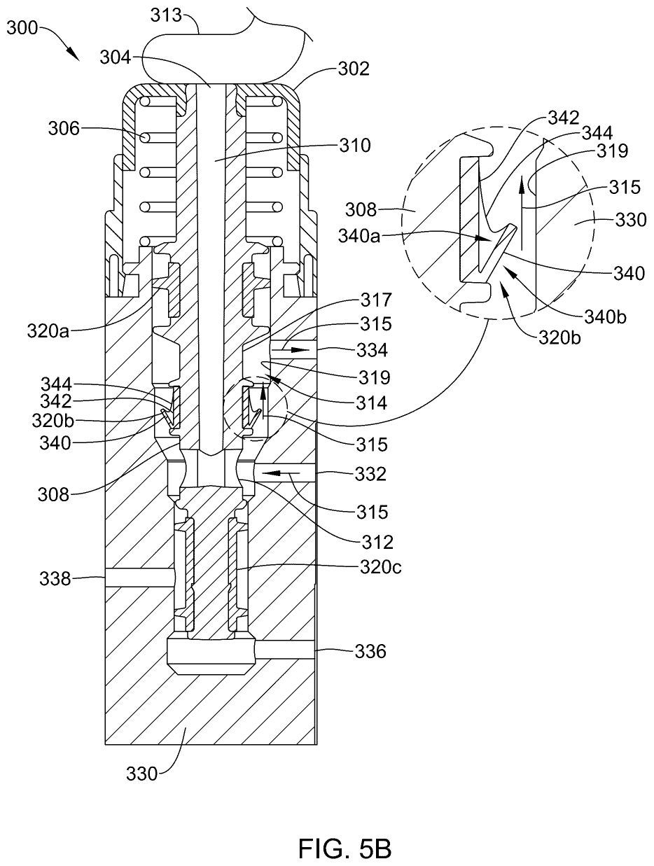

FIGS. 5A and 5B depict an illustrative configuration of the second seal 320b positioned circumferentially around the valve stem 308 having or forming an elongate body. The second seal 320b may have a flange 340 extending radially outward from a body portion 342 (e.g., an elongate body, a cylindrical elongate body, and/or other suitable body configuration) to the inner surface 319 of the valve body 330 and circumferentially around the body portion 342. In some examples, the body portion 342 of the second seal 320b may be coaxial with and/or coupled with the elongate body of the valve stem 308. One or more ribs 344 may extend between the flange 340 and the body portion 342.

As depicted in FIG. 5A, the one or more ribs 344 may be tensioned and/or taut and support the flange 340 in an expanded or taut position (e.g., the ribs 344 may restrict deformation of the flange 340) when the valve 300 is in the first configuration and the pressure proximal of the flange 340 (at a first side 340a of the flange 340) of the second seal 320b is greater than a pressure distal of the flange 340 (e.g., at a second side 340b of the flange 340) such that insufflation backflow from the gas outlet passage 334 in the direction of arrow 321, if any, does not cross the second seal 320b, which facilitates maintaining a desired insufflation. In some examples, the flange 340 may be positioned perpendicular to or approximately perpendicular to a longitudinal axis of the valve 300 and the ribs 344 may be configured to maintain the flange 340 in such a position when the valve 300 is in the first configuration with gas directed through the central lumen 310.

As depicted in FIG. 5B, one or more of the ribs 344 may be configured to be and/or may be flexed and/or compressed (e.g., deformed) such that the flange 340 may be folded or flexed or otherwise deformed to allow gas to cross the second seal 320b and travel along a fluid pathway shown by arrows 315 from the gas inlet passage 332 to the gas outlet passage 334 when the valve 300 is in the second configuration and pressure distal of the flange 340 (e.g., at the second side 340b of the flange 340) is greater than the pressure proximal of the flange 340 (e.g., at the first side 340a of the flange 340). A configuration of the flange 340 and/or the ribs 344 may determine how the flange 340 adjusts or otherwise deforms when the valve 300 is in the second position.

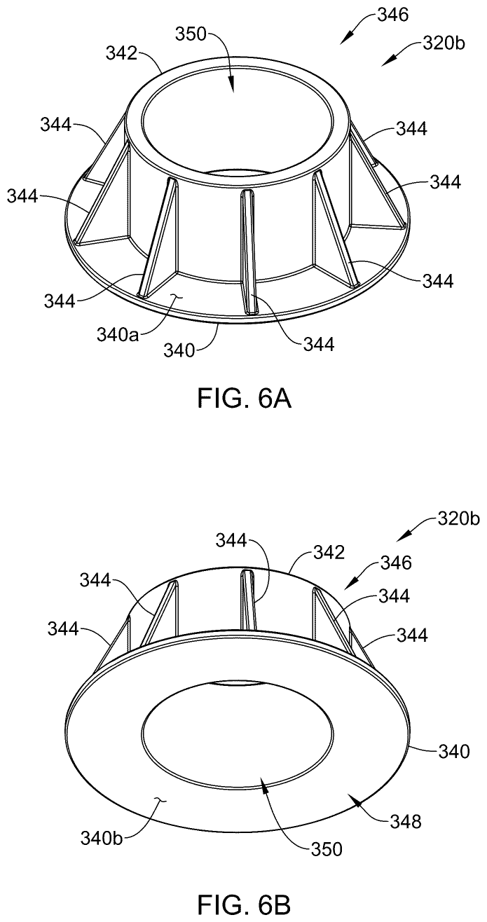

FIGS. 6A and 6B depict the second seal 320b depicted in FIGS. 5A and 5B in the expanded or taut (e.g., sealing) position or configuration. FIG. 6A schematically depicts a perspective view of the second seal 320b from a first side 346 (e.g., a proximal side) of the second seal 320b. FIG. 6B schematically depicts a perspective view of the second seal 320b from a second side 348 (e.g., a distal side) of the second seal 320b.

The second seal 320b may include one or more flanges 340, one or more body portions 342, and one or more ribs 344. In some examples and as depicted in FIGS. 6A and 6B, the body portion 342 may be cylindrical and have an inner surface and an outer surface. In one example, the inner surface of the body portion 342 may define an inner lumen 350, which may be configured to receive the valve stem 308.

As discussed, the one or more ribs 344 may extend between the flange 340 and the body portion 342. In some examples, the flange 340 may have a first side 340a and a second side 340b opposing the first side 340a and the one or more ribs 344 may extend between the first side 340a of the flange 340 and the outer surface of the body portion 342. In one example, one or more ribs 344 may extend from a location proximate a proximal end of the body portion 342, along an outer surface of the body portion 342 to a location at which the outer surface of the body portion 342 meets the first side 340a of the flange, radially outward to a location proximate an outer circumference of the flange 340, and diagonally between the location proximate the proximal end of the body portion 342 and the location proximate an outer circumference of the flange 340, but other suitable configurations are contemplated.

The flange 340 and/or the ribs 344 may be configured in any suitable manner. In some examples, configuring the flange 340 and/or the ribs 344 to cause the flange 340 to deform in a desired manner may result in a consistent, predictable deformation of the flange 340 and consistent insufflation performance with minimal turbulence.

The components of the second seal 320b may be formed from any suitable material. For example, the components of the second seal 320b may be formed from one or more materials including, but not limited to, a polymer, thermoplastic elastomers (TPEs), thermoplastic polyurethane (TPU), liquid silicone rubber (LSR), and/or other suitable materials.

The components of the second seal 320b may be formed from a material having any suitable durometer. In one example, the material of the second seal 320b may have a durometer in a range of about 20-80 shore A, about 30-60 shore A, and/or other suitable values within one or more other suitable ranges of durometer, but could be softer or firmer depending on the geometry used for the second seal 320b and the amount of interference desired with the inner surface 319 of the valve body 330. In one example, the second seal 320b may be formed from silicone with a durometer in a range of 40-50 shore A, but this is not required.

The flange 340 may have any suitable configuration configured to maintain the flange 340 in the expanded or taut position (e.g., as depicted in FIGS. 5A, 6A, and 6B) when the valve 300 is in the first configuration with a pressure at the first side 340a of the flange 340 greater than at the second side 340b and adjust to a folded or flexed position when the pressure at the first side 340a of the flange 340 is less than at the second side 340b. For example, the flange 340 may be formed from a material and may have a thickness configured to facilitate the flange 340 adjusting to a position suitable for blocking fluid when in the expanded or taut position and to adjust by bending, folding, flexing, and/or otherwise deforming in a desired manner when in the folded or flexed position to allow fluid to pass the second seal 320b. Examples of such suitable materials include, but are not limited to, polymers, thermoplastic elastomers (TPEs), thermoplastic polyurethanes (TPUs), liquid silicone rubbers (LSRs), and/or other suitable materials. Examples of such suitable thicknesses include, but are not limited to, thicknesses in a range of about 0.254 millimeters (mm) to about 1.27 mm (e.g., about 0.010 inches to about 0.050 inches). In some examples, the folded or flexed position of the flange 340 may be configured to allow a gas to cross the second seal 320b at a desired flow rate.

The one or more ribs 344 may have any suitable configuration (e.g., a suitable flexibility or rigidity, etc.) configured to maintain the flange 340 in a desired expanded or taut position (e.g., in a sealing position) when the one or more ribs 344 are under tension and to allow the flange 340 to flex, bend, fold, etc. when the one or more ribs 344 are under compression. In some examples, the ribs 344 may be sized to maintain the flange 340 in a position or configuration that is perpendicular to the longitudinal axis of the valve 300. In some examples, one or more of the ribs 344 may form a solid volume between the body portion 342 and the flange 340, one or more of the ribs 344 may include one or more cut outs or openings, and/or one or more of the ribs 344 may have other suitable configurations to achieve a desired flexibility or rigidity and facilitate forming a desired shape and/or configuration when under tension (e.g., a tension force when a pressure on the first side 340a of the flange is greater than a pressure on the second side 340b of the flange 340) and/or when under compression (e.g., a compressive force when a pressure on the second side 340b of the flange 340 is greater than a pressure on the first side 340a of the flange 340). Further, the ribs 344 may have one or more thicknesses and/or rigidities that facilitate forming desired shapes and/or configurations when under compression and/or tension. In some examples, when the ribs 344 are formed from the materials discussed herein with the durometers discussed herein, suitable thicknesses of the ribs 344 to achieve the desired shapes and/or configurations when under compression and/or tension may include, but are not limited to, thicknesses in a range of about 0.127 mm to about 0.762 mm (e.g., about 0.005 inches to about 0.030 inches).

Any suitable number of ribs 344 circumferentially spaced about the body portion 342 and/or the flange 340 may be utilized such that the flange 340 may be maintained in the desired sealing position and may adjust to a desired folded or flexed position. In some examples, the number of ribs 344 may be an even number or an odd number and may include, but are not limited to, one rib 344, two ribs 344, three ribs 344, six ribs 344, nine ribs 344, twelve ribs 344, and/or other suitable number of ribs 344. In some examples, when two or more ribs 344 are utilized, the ribs may be equally or unequally spaced circumferentially around an outer circumference of the body portion 342. In some examples, a number of ribs 344 included in the second seal 320b and a spacing of those ribs 344 may be selected to facilitate allowing a desired rate of gas flow across the second seal 320b when the valve 300 is in the second configuration.

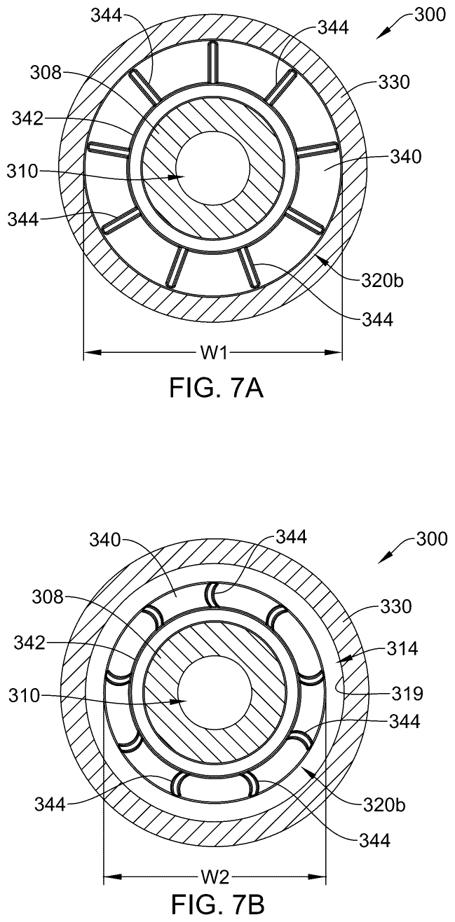

FIGS. 7A and 7B depict schematic cross-section views of the valve 300 taken at an axial location proximal of the second seal 320b. FIG. 7A depicts a schematic cross-section view of the valve 300 when the valve 300 is in the first configuration with the flange 340 of the second seal 320b in the expanded or taut position and having a first width W1 (e.g., a first diameter). FIG. 7B depicts a schematic cross-section view of the valve 300 when the valve 300 is in the second configuration with the flange 340 in the folded or flexed position and having a second width W2 (e.g., a second diameter) that is less than the first width W1.

All of the ribs 344 depicted in FIG. 7A may have a same configuration (e.g., material, thickness, shape, volume, etc.), the ribs 344 may be equally spaced circumferentially around the body portion 342, and the flange 340 may have a uniform configuration. Although other suitable number of ribs 344 may be utilized, the second seal 320b may include nine ribs 344.

When the ribs 344 have the same configuration, the ribs 344 are equally spaced circumferentially around the body portion 342, and the flange 340 has a uniform configuration, the space 314 at the outer circumference of the flange 340 in the flexed or folded position (e.g., when the valve is in the second configuration) may have a uniform or substantially uniform width W2 circumferentially around an outer perimeter of the flange 340, as depicted for example in FIG. 7B. In some examples, the number of the ribs 344 and/or configuration of the ribs 344 utilized may be adjusted to increase or decrease the width of the space 314 circumferentially around the outer perimeter of the flange 340 and, respectively, increase or decrease a flow rate of gas across the second seal 320b.

As discussed, a configuration of the ribs 344 and/or a configuration of the flange 340 of the second seal 320b may be adjusted or configured to adjust how the flange 340 deforms when the valve 300 is in the second configuration. For example, to create a desired flex or fold or deforming of the flange 340, the flange 340 may be configured to have desired flexibilities at different circumferential locations, a desired number of ribs 344 circumferentially spaced about the body portion 342 may be selected, one or more of the ribs 344 may have a different thickness than one or more other ribs 344, the ribs 344 may or may not be equally spaced circumferentially around the body portion 342, and/or one or more other features of the flange 340 and/or the ribs 344 may be adjusted to achieve a desired flex or fold or deforming of the flange 340.

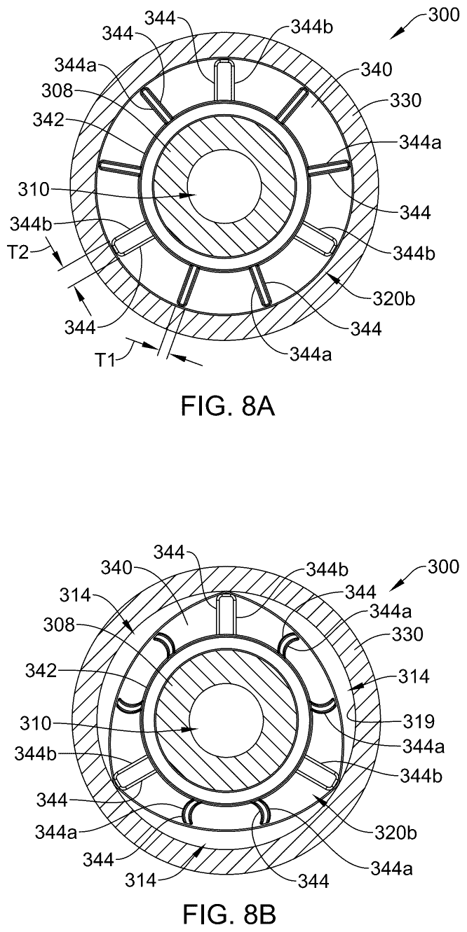

FIGS. 8A and 8B depict a schematic cross-section view of the valve 300 taken at an axial location proximal of the second seal 320b, where one or more of the ribs 344 have been configured to have a different thickness than one or more other ribs 344 to achieve a desired flow profile of gas (e.g., a desired maximum flow rate of gas, a desired laminarity of flow, etc.) across the second seal 320b. FIG. 8A depicts a schematic cross-section view of the valve 300 when the valve 300 is in the first configuration with the second seal 320b in the expanded or taut position. FIG. 8B depicts a schematic cross-section view of the valve 300 when the valve 300 is in the second configuration with the second seal 320b in the folded or flexed position.

A first set 344a of ribs 344 may be configured to flex or fold or deform to allow the flange 340 to flex or fold or deform when the valve 300 is in the second configuration with a pressure at the second side 340b of the flange 340 greater than a pressure at the first side 340a of the flange 340 and a second set of ribs 344 may be configured to remain taut when the valve 300 is in the second configuration. In some examples, to achieve a desired rigidity or flexibility of the ribs 344, the first set 344a of the ribs 344 extending between the flange 340 and the body portion 342 may have a first thickness T1 and the second set 344b of the ribs 344 extending between the flange 340 and the body portion 342 may have a second thickness T2 that is thicker than the first thickness T1, as depicted for example in FIG. 8A. In some examples, the ribs 344 of the first set 344a may have a first rigidity due to having the first thickness T1 that is less than (e.g., more flexible than) a second rigidity of the ribs 344 of the second set 344b having the second thickness T2 (e.g., the second rigidity may be more rigid than the first rigidity). The rigidity of the ribs 344 may be modified in one or more other suitable manners including, but not limited to, by adjusting a volume of material in one or more ribs 344, adjusting a material of one or more ribs 344, and/or by adjusting one or more other suitable characteristics of the ribs 344. Other suitable configurations of the ribs 344 are contemplated.

Other than the thicknesses of the ribs 344 and the resulting volume of the ribs 344, the ribs 344 of the first set 344a and the second set 344b depicted in FIG. 8A may have a same configuration, the ribs 344 may be equally spaced circumferentially around the body portion 342, and the flange 340 may have a uniform configuration. In some example configurations, the first set 344a of ribs 344 may have more ribs 344 than the second set 344b, but other suitable configurations are contemplated. Although other suitable number of ribs 344 may be utilized, the second seal 320b may include nine ribs 344, where the first set 344a may have more ribs 344 (e.g., six ribs, etc.) than the second set 344b (e.g., three ribs 344). In one example configuration of the position of the ribs 344 of the second seal 320b, the ribs 344 of the second set 344b may be equally spaced circumferentially around the body portion 342 of the second seal 320b and an equal number of ribs 344 from the first set 344a may be located between each set of two circumferentially sequential ribs 344 from the second set 344b and equally spaced between the set of two circumferentially sequential ribs 344, as depicted for example in FIG. 8A. In another example configuration of the position of the ribs 344 of the second seal 320b, the first set 344a may include nine ribs 344 and the second set 344b may include three ribs 344, where the ribs 344 of the second set 344b may be equally spaced circumferentially around the body portion 342 of the second seal 320b and three ribs 344 from the first set 344a may be located between each set of two circumferentially sequential ribs 344 from the second set 344b and equally spaced between the set of two circumferential sequential ribs 344. Other suitable configurations of the positioning of the ribs 344 are contemplated.

Utilizing ribs 344 with different thicknesses or rigidities may facilitate providing the second seal 320b with a desired shape when the valve 300 is in the second configuration to facilitate allowing gas to pass from the gas inlet passage 332 to the gas outlet passage 334 in a desired manner (e.g., at a desired flow rate, at desired locations, with a desired flow profile, etc.) For example, when the second set 344b includes three ribs 344 that are configured to remain taut when the valve 300 is in the second configuration, three zones of deformation of the flange 340 may be achieved (e.g., three zones where the space 314 crosses the second seal 320b are achieved) along the flange 340 proximate ribs 344 from the first set 344a that are configured to fold or flex or deform when the valve 300 is in the second condition and pressure at the second side 340b of the flange 340 is greater than pressure at the first side 340a of the flange 340, as depicted for example in FIG. 8B, where the each zone of deformation may be located between two circumferentially sequential ribs 344 from the second set 344b at which the flange 340 may remain taut and/or contacting the valve body 330. In some examples, configuring the flange 340 and/or the ribs 344 to cause the flange 340 to deform at one or more desired locations may result in achieving a desired deformation of the flange 340, a desired flow turbulence, and a desired insufflation performance.

Although the configurations of the flange 340, the body portion 342, and the ribs 344 were discussed with respect to the second seal 320b of a gas/water valve 300, similar configurations may be utilized with other seals of the valve 300 and/or with other valves. For example, the configurations of the flange 340, the body portion 342, and/or the ribs 344 may be utilized in the first seal 320a and/or the third seal 320c of the valve 300.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A valve stem for a medical device, the valve stem comprises:
an elongate body;
a first opening in the elongate body;
a second opening in the elongate body;
a lumen extending between the first opening and the second opening; and
a seal extending circumferentially around the elongate body, the seal having a body portion, a flange extending circumferentially around the elongate body and radially outward from the elongate body, and one or more ribs extending between the body portion and the flange.

2. The valve stem of claim 1, wherein the seal comprises a plurality of ribs equally spaced circumferentially around the body portion of the seal.

3. The valve stem of claim 1, wherein:
the flange has a first side and a second side opposing the first side and the one or more ribs extend between the body portion and the first side of the flange, and
the one or more ribs are configured to be in tension and maintain the flange in a taut configuration when a pressure at the first side of the flange is greater than a pressure at the second side of the flange.

4. The valve stem of claim 3, wherein the one or more ribs are configured to flex and allow the flange to deform when the pressure at the second side of the flange is greater than the pressure at the first side of the flange.

5. The valve stem of claim 1, wherein the one or more ribs comprise:
a first set of one or more ribs each having a first rigidity; and
a second set of one or more ribs each having a second rigidity that is more rigid than the first rigidity.

6. The valve stem of claim 5, wherein the first set of one or more ribs has more ribs than the second set of one or more ribs.

7. The valve stem of claim 5, wherein the second set of one or more ribs has a plurality of ribs equally spaced circumferentially around the body portion of the seal and the first set of one or more ribs has a plurality of ribs equally divided between circumferentially sequential ribs of the second set of one or more ribs.

8. The valve stem of claim 7, wherein:
the flange has a first side and a second side opposing the first side and the first set of one or more ribs and the second set of one or more ribs extend between the body portion and the first side of the flange, and
the ribs of the first set of one or more ribs are configured to flex and allow the flange to deform between the sequential ribs of the second set of one or more ribs when a pressure at the second side of the flange is greater than a pressure at the first side of the flange.

9. A seal for a valve stem of a medical device, the seal comprising:
a body portion defining a lumen configured to receive the valve stem;
a flange extending circumferentially around the body portion; and
a plurality of ribs extending between the body portion and the flange.

10. The seal of claim 9, wherein the plurality of ribs are equally spaced circumferentially around the body portion.

11. The seal of claim 9, wherein each rib of the plurality of ribs has a rigidity configured to maintain the flange in a taut configuration when a pressure at a first side of the flange is greater than a pressure at a second side of the flange and allow the flange to deform when the pressure at the second side of the flange is greater than the pressure at the first side of the flange.

12. The seal of claim 9, wherein the plurality of ribs comprise:
a first set of one or more ribs each having a first rigidity; and
a second set of one or more ribs each having a second rigidity that is more rigid than the first rigidity.

13. The seal of claim 12, wherein the first set of one or more ribs has more ribs than the second set of one or more ribs.

14. The seal of claim 12, wherein the second set of one or more ribs has a plurality of ribs equally spaced circumferentially around the body portion of the seal and the first set of one or more ribs has a plurality of ribs equally divided between circumferentially sequential ribs of the second set of one or more ribs.

15. The seal of claim 14, wherein:
the flange has a first side and a second side opposing the first side and the first set of one or more ribs and the second set of one or more ribs extend between the body portion and the first side of the flange, and
the ribs of the first set of one or more ribs are configured to flex and allow the flange to deform between the ribs of the second set of one or more ribs when a pressure at the second side of the flange is greater than a pressure at the first side of the flange.

16. The seal of claim 12, wherein:
the first set of one or more ribs has nine ribs,
the second set of one or more ribs has three ribs equally spaced circumferentially about the body portion, and
three ribs of the first set of one or more ribs are positioned between each two circumferentially sequential ribs of the second set of one or more ribs.

17. A valve assembly for a medical device, comprising:
a valve body having a gas inlet passage and a gas outlet passage;
a valve cap proximate a proximal end of the valve body;
a valve stem connected to the valve cap and configured to translate within the valve body between an upper position and a lower position, the valve stem comprising an elongate body and a central lumen extending through the elongate body between a first opening in the elongate body and a second opening in the elongate body; and
a seal extending around the valve stem, the seal having a flange and one or more ribs supporting the flange in a flow blocking configuration between the gas inlet passage and the gas outlet passage.

18. The valve assembly of claim 17, wherein:
the flange has a first side and a second side,
the one or more ribs extend between a body portion of the flange and the first side,
the one or more ribs are configured to support the flange in the flow blocking configuration when a pressure on the first side of the flange is greater than a pressure on the second side of the flange, and
the one or more ribs are configured to flex to allow the flange to adjust to a flow passing configuration when a pressure on the second side of the flange is greater than a pressure on the first side of the flange.

19. The valve assembly of claim 17, wherein the one or more ribs comprise:
at least one rib with a first rigidity; and
at least one rib with a second rigidity that is more rigid than the first rigidity.

20. The valve assembly of claim 17, wherein the seal is located at a position along the valve stem that is configured to be maintained between the air inlet passage and the air outlet passage of the valve body during operation of the medical device.
